(19) United States
(12) Patent Application Publication
(10) Pub. No.: US 2023/0172945 A1
(43) Pub. Date: Jun. 8, 2023

(54) APPLICATION OF COMPOUNDS IN CONTROLLING OR KILLING MITES

(71) Applicant: SMILEBIOTEK ZHUHAI LIMITED, Zhuhai, Guangdong (CN)

(72) Inventor: Yan ZHANG, Zhuhai, Guangdong (CN)

(21) Appl. No.: 17/920,731

(22) PCT Filed: Apr. 23, 2021
(86) PCT No.: PCT/CN2021/089105

§ 371 Date: Oct. 21, 2022

(30) Foreign Application Priority Data
Apr. 24, 2020 (CN) 202010334025.0

(51) Int. Cl. A61K 31/56; A61K 31/7048; A61K 8/60; A61K 31/352; A61K 8/49; A61K 8/63; A61Q 17/02

(52) U.S. Cl. CPC A61K 31/56; A61K 31/7048; A61K 8/602; A61K 31/352; A61K 8/498; A61K 8/63; A61Q 17/02; A61K 2800/70

(57) ABSTRACT

An application of a series of compounds in controlling or killing mites. It has been found by comparison that the compounds, when compared with a control group, can reduce the survival time of mites; in particular, taraxerol, diosmetin, and taraxasteryl acetate can significantly shorten the survival time of mites, and can be used for miticide and mite control products (such as drugs, cosmetic products, daily products and so on).

### TECHNICAL FIELD

The present invention relates to the field of pharmaceuticals, and in particular to use of a compound having a structure of formula I, II or III in controlling or killing mites.

### BACKGROUND

Mites are a class of tiny animals belonging to Arthropoda, Arachnida, Latigastra, generally having a body size of about 0.5 mm, some as small as 0.1 mm, and most species have a body size of less than 1 mm. It is found that mites are closely related to the health status of human beings and animals (such as dogs, cats and other companion animals). For example, mites such as gamasid mites (Mesostigmata spp.), chiggers, itch mite (Sarcoptes scabiei), Demodex mites (Demodex spp.), flour mites (Acarus siro), dust mite and Pyemotes spp. are hematophagic and may harm the skin, causing “acne rosacea” or demodicosis, hypersensitivity, urinary acariasis, pulmonary acariasis, intestinal acariasis, scabies and the like, which seriously endanger the health of human beings and animals. Demodex mites are microscopic ectoparasites that usually affect the pilosebaceous unit of the skin. Among the reported species, D. folliculorum and D. brevis have been found on the body surface of humans, and mainly reside in the hair follicles, sebaceous glands and meibomian glands. Demodex mites feed on epithelial cells of hair follicle, causing follicle dilatation and hair loss, manifesting clinically as blepharitis marginalis, meibomian gland dysfunction, madarosis, abnormal eyelash alignment, conjunctivitis and blepharoconjunctivitis, pterygium, keratitis, basal cell carcinoma of eyelid and the like. Demodex mites may also feed on lipids, causing xerophthalmia. In addition, Demodex mites may also cause mechanical obstruction of meibomian gland ducts, causing difficulty in lipid excretion and excessive retention of secretions, leading to the formation of chalazion. The clinical manifestations of the ocular diseases caused by Demodex infestation mainly include: recurrent red and itch eyes, dry eyes, burning eyes, foreign body sensation, photophobia and increased secretion; the diseases may also be accompanied by recurrent eyelash loss; blurred vision and decreased vision may occur in severe cases affecting the cornea. In addition to the above ocular diseases, a number of studies in recent years have reported that Demodex infestation is associated with a variety of common skin diseases, including seborrhoeic dermatitis, acne, acne rosacea, pityriasis folliculorum, perioral dermatitis, demodicosis, basal cell carcinoma and the like (see, e.g., Luo, X., Li, J., Chen, C., Tseng, S. & Liang, L, Ocular Demodicosis as a Potential Cause of Ocular Surface Inflammation, Cornea 36 Suppl 1, s9-s14; Karincaoglu, Y., Tepe, B., Kalayci, B., Atambay, M. & Seyhan, M, Is Demodex folliculorum an aetiological factor in seborrhoeic dermatitis?, Clinical and experimental dermatology 34, e516-520; Chen, W. & Plewig, G, Human demodicosis: revisit and a proposed classification, The British journal of dermatology 170, 1219-1225).

In particular, reports where the association of mites with ocular diseases has been confirmed include:

Human Permanent Ectoparasites; Recent Advances on Biology and Clinical Significance of Demodex Mites: Narrative Review Article (Iranian journal of parasitology 12(1):12-21) has shown that D. brevis lives in the sebaceous glands and meibomian glands and D. folliculorum often occupies the hair follicle area of human eyelashes, of which both will cause ocular diseases after infecting the facial skin.

Ocular Demodicosis as a Potential Cause of Ocular Surface Inflammation (Cornea 36 Suppl 1(Suppl 1):s9-s14) demonstrates that D. brevis and D. folliculorum are two Demodex species that cause ocular demodicosis in humans, and there is a positive correlation between human age and the risk of the disease and a strong positive correlation between ocular demodicosis and ocular surface inflammatory conditions.

Demodex species in human ocular disease: new clinicopathological aspects (International ophthalmology 37(1):303-312) has shown that D. brevis and D. folliculorum are associated with the pathogenesis of external ocular diseases, and ocular demodicosis can cause an imbalance in the extraocular environment.

Ocular Demodex: a systematic review of the clinical literature (Ophthalmic & physiological optics: the journal of the British College of Ophthalmic Opticians (Optometrists) 40(4):389-432) has shown that Demodex infestation is a potential cause of several ocular surface diseases, and the parasitism of Demodex mites on the anterior structures of the eyes such as eyelids, eyelashes and the ocular surface can lead to the ocular demodicosis in humans, and the incidence is positively correlated with age.

The relationship between demodex and ocular discomfort (Investigative ophthalmology & visual science 51(6):2906-2911) suggested that the number of Demodex mites is positively correlated with the severity of ocular diseases and the age of patients.

Demodex mites (Clinics in dermatology 32(6):739-743) suggested that Demodex infestation can cause chronic blepharitis marginalis, and the prevalence of chronic blepharitis marginalis is positively correlated with the number of mites and the age of patients.

Quantitative Analysis of the Bacteria in Blepharitis With Demodex Infestation (Frontiers in microbiology 9:1719) has shown that D. brevis and D. folliculorum infestations can lead to the occurrence of ocular diseases, the prevalence is positively correlated with the number of mites and the age of patients, and the Demodex mites are carriers of Bacillus spp., which can play a role as co-pathogens in the pathogenesis of blepharitis marginalis.

Bacillus oleronius and Demodex mite infestation in patients with chronic blepharitis (Clinical microbiology and infection: the official publication of the European Society of Clinical Microbiology and Infectious Diseases 18(10):1020-1025) suggested that Demodex mites may cause the occurrence of blepharitis marginalis and are carriers of Bacillus spp., which can play a role as co-pathogens in the development of blepharitis marginalis.

Correlation between ocular Demodex infestation and serum immunoreactivity to Bacillus proteins in patients with Facial rosacea (Ophthalmology 117(5):870-877.e871) suggested that the incidence of ocular Demodex infestation in patients with xerophthalmia is lower than that in patients without xerophthalmia, and that the mite infestation and bacterial infection can coexist in ocular surface inflammation.

High prevalence of demodex brevis infestation in chalazia (American journal of ophthalmology 157(2):342-348.e341) has shown that the incidence of demodicosis caused by D. brevis is high in adults and pediatric patients with chalazion, and the ocular demodicosis is a major cause of chalazion.

Diseases caused by mites (such as ocular diseases and skin diseases) have not yet received sufficient clinical attention, and are often misdiagnosed as bacterial diseases and the like, while the conventional antibacterial treatments usually have little curative effect.

Therefore, the present invention provides a series of compounds for use in controlling or killing mites.

### SUMMARY

The present invention provides use of a compound of formula I or a salt, an isomer or a solvate thereof in preparing a product for controlling or killing mites, wherein the compound of formula I is:

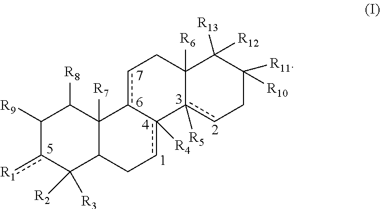

Wherein, R₁ is selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₂₋₈ alkynyl, CH═CH₂, CH═CH(C₁₋₈ alkyl), C(C₁₋₈ alkyl)=CH₂, C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂, or when the bond between R₁ and the carbon atom at position 5 is a double bond, R₁ is selected from O and S.

R₂, R₃, R₆, R₇, R₈ and R₉ are independently selected from H, C₁₋₈ alkyl, O(C₁₋₈ alkyl), NH(C₁₋₈ alkyl), N(C₁₋₈ alkyl)₂, C₃₋₁₁ cycloalkyl, O(C₁₋₈ cycloalkyl), OH and NH₂.

R₄ is selected from H, C₁₋₈ alkyl, O(C₁₋₈ alkyl), NH(C₁₋₈ alkyl), N(C₁₋₈ alkyl)₂, C₃₋₁₁ cycloalkyl, O(C₁₋₈ cycloalkyl), OH and NH₂, or when the bond between carbon atoms at positions 1 and 4 is a double bond, R₄ is absent.

R₅ is selected from H, C₁₋₈ alkyl, O(C₁₋₈ alkyl), NH(C₁₋₈ alkyl), N(C₁₋₈ alkyl)₂, C₃₋₁₁ cycloalkyl, O(C₁₋₈ cycloalkyl), OH and NH₂, or when the bond between carbon atoms at positions 2 and 3 is a double bond, R₅ is absent.

The bond between carbon atoms at positions 6 and 7 is a double bond or single bond.

R₁₀ and R₁₃ are independently selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₁₋₈ alkynyl, CH═CH(C₁₋₈ alkyl), CH═CH₂, C(C₁₋₈ alkyl)=CH₂, C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂.

R₁₁ and R₁₂ are independently selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₁₋₈ alkynyl, CH═CH(C₁₋₈ alkyl), C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂, or, R₁₁ and R₁₂ are carbon atoms and together with carbon atoms therebetween form a 5- to 8-membered ring, wherein H on any carbon atom on the ring may be substituted with C₁₋₈ alkyl, O(C₁₋₈ alkyl), NH(C₁₋₈ alkyl), N(C₁₋₈ alkyl)₂, C₃₋₁₁ cycloalkyl, O(C₁₋₈ cycloalkyl), OH, NH₂, C₂₋₈ alkenyl, C₂₋₈ alkynyl, CH═CH₂, C(C₁₋₈ alkyl)=CH₂, CH═CH(C₁₋₈ alkyl), C(C₁₋₈ alkyl)═CH(C₁₋₈ alkyl) or C(C₁₋₈ alkyl)═C(C₁₋₈ alkyl)₂.

Specifically, the compound of formula I described herein is a compound of formula I-1:

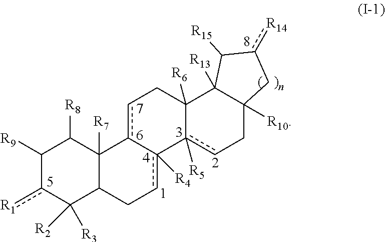

Wherein, when the bond between R₁₄ and the carbon atom at position 8 is a single bond, R₁₄ is selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₂₋₈ alkynyl, CH═CH(C₁₋₈ alkyl), C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂, and hydrogen atom on the carbon atom at position 8 is optionally substituted with R₁₄; when the bond between R₁₄ and the carbon atom on position 8 is a double bond, R₁₄ is selected from O, S, CH₂, CH(C₁₋₈ alkyl) and C(C₁₋₈ alkyl)₂.

R₁₅ is selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₂₋₈ alkynyl, CH═CH(C₁₋₈ alkyl), C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂.

n is an integer of 1-4, such as 1, 2, 3 or 4.

Specifically, R₁ is selected from H, C₁₋₃ alkyl, OH, OC(═O)(C₁₋₃ alkyl), (C₁₋₃ alkyl)CO₂H, CF₃, CHF₂ and CH₂F, or when the bond between R₁ and the carbon atom at position 5 is a double bond, R₁ is O. More specifically, R₁ is selected from OH and OC(═O)(C₁₋₃ alkyl). Still more specifically, R₁ is selected from OH and OC(═O)CH₃.

Specifically, R₂, R₃, R₆, R₇, R₈ and R₉ are independently selected from H, C₁₋₃ alkyl, O(C₁₋₃ alkyl), C₃₋₆ cycloalkyl, OH and NH₂. Still more specifically, R₂, R₃, R₆, R₇, R₈ and R₉ are independently selected from H and methyl.

Specifically, the bond between carbon atoms at positions 1 and 4 is a single bond, and R₄ is selected from H, C₁₋₃ alkyl, O(C₁₋₃ alkyl), C₃₋₆ cycloalkyl, O(C₁₋₃ cycloalkyl) and OH. More specifically, the bond between carbon atoms at positions 1 and 4 is a single bond, and R₄ is selected from H and methyl.

Specifically, the bond between carbon atoms at positions 2 and 3 is a single bond, and R₅ is selected from H, C₁₋₃ alkyl, O(C₁₋₃ alkyl), C₃₋₆ cycloalkyl, O(C₁₋₃ cycloalkyl) and OH. More specifically, the bond between carbon atoms at positions 2 and 3 is a single bond, and R₅ is selected from H and methyl.

Specifically, the bond between carbon atoms at positions 6 and 7 is a single bond.

Specifically, R₁₀ and R₁₃ are independently selected from H, C₁₋₃ alkyl, O(C₁₋₃ alkyl), C₃_6 cycloalkyl, OH and NH₂. More specifically, R₁₀ and R₁₃ are independently selected from H and methyl.

Specifically, when the bond between R₁₄ and the carbon atom at position 8 is a single bond, R₁₄ is selected from H, C₁₋₃ alkyl, O(C₁₋₃ alkyl), C₃₋₆ cycloalkyl, OH and NH₂, and similarly, the hydrogen atom on the carbon atom at position 8 is substituted with R₁₄; when the bond between R₁₄ and the carbon atom at position 8 is a double bond, R₁₄ is selected from CH₂, CH(C₁₋₃ alkyl) and C(C₁₋₃ alkyl)₂. More specifically, when the bond between R₁₄ and the carbon atom at position 8 is a single bond, R₁₄ is selected from H and methyl, and similarly, the hydrogen atom on the carbon atom at position 8 is substituted with H or methyl; when the bond between R₁₄ and the carbon atom at position 8 is a double bond, R₁₄ is CH₂ (methylene).

Specifically, R₁₅ is selected from H, C₁₋₃ alkyl, O(C₁₋₃ alkyl), C₃₋₆ cycloalkyl, OH, NH₂, CH═CH(C₁₋₃ alkyl), C(C₁₋₃ alkyl)=CH₂, C(C₁₋₃ alkyl)═CH(C₁₋₃ alkyl) and C(C₁₋₃ alkyl)═C(C₁₋₃ alkyl)₂. More specifically, R₁₅ is selected from H, C₁₋₃ alkyl and C(CH₃)=CH₂.

In a specific embodiment of the present invention, the compound of formula I is taraxasterol, taraxerol, taraxerone, roburic acid, taraxasterol acetate, taraxeryl acetate, lupenone, or a salt, an isomer or a solvate thereof.

The present invention further provides use of a compound of formula II or a salt, an isomer or a solvate thereof in preparing a product for controlling or killing mites, wherein the compound of formula II is:

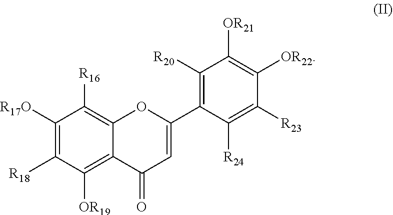

Wherein, R₁₆, R₁₈, R₂₀, R₂₃ and R₂₄ are independently selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₂₋₈ alkynyl, CH═CH(C₁₋₈ alkyl), C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂.

R₁₇, R₁₉, R₂₁ and R₂₂ are independently selected from H, C₁₋₈ alkyl, O(C₁₋₈ alkyl), C₃₋₁₁ cycloalkyl, aryl, heteroaryl, C₃₋₁₁ heterocycloalkyl, O(C₁₋₈ cycloalkyl) and a group of formula II-1, II-2 or II-3:

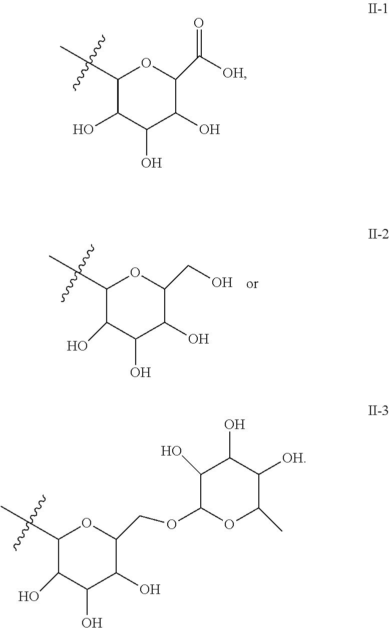 [SMILES: *C1OC(C(=O)O)C(O)C(O)C1O ; *C1OC(CO)C(O)C(O)C1O ; *C1OC(COC2OC(C)C(O)C(O)C2O)C(O)C(O)C1O]

Specifically, R₁₆, R₁₈, R₂₀, R₂₃ and R₂₄ are independently selected from H, halogen, C₁₋₃ alkyl, O(C₁₋₃ alkyl), NH(C₁₋₃ alkyl), N(C₁₋₃ alkyl)₂, C₃₋₁₁ cycloalkyl, OH, NH₂, OC(═O)(C₁₋₃ alkyl), C(═O)(C₁₋₃ alkyl), (C₁₋₃ alkyl)CO₂H, CO₂H, CN, CF₃, CHF₂, CH₂F, NO₂, C₂₋₃ alkenyl, C₂₋₃ alkynyl, CH═CH(C₁₋₃ alkyl), C(C₁₋₃ alkyl)═CH(C₁₋₃ alkyl) and C(C₁₋₃ alkyl)═C(C₁₋₃ alkyl)₂. More specifically, R₁₆, R₁₈, R₂₀, R₂₃ and R₂₄ are independently selected from H and methyl.

Specifically, R₁₇, R₁₉, R₂₁ and R₂₂ are independently selected from H, C₁₋₃ alkyl, O(C₁₋₃ alkyl) and a group of formula II-1 or II-2. More specifically, R₁₇, R₁₉, R₂₁ and R₂₂ are independently selected from H and methyl.

In a specific embodiment of the present invention, the compound of formula II is diosmetin or a salt, an isomer or a solvate thereof.

The present invention further provides use of a compound of formula III or a salt, an isomer or a solvate thereof in preparing a product for controlling or killing mites, wherein the compound of formula III is:

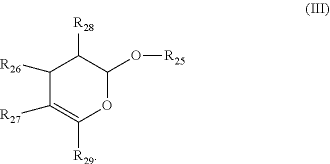

Wherein, R₂₅ is selected from

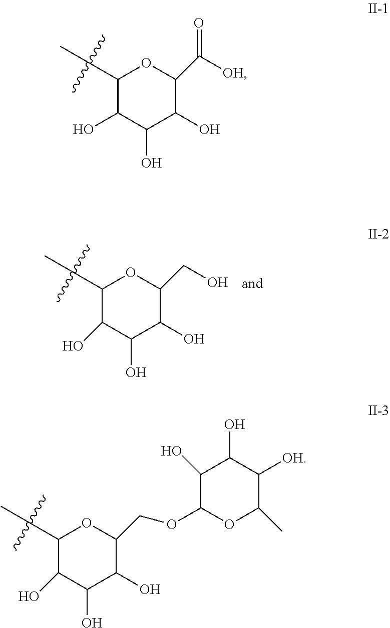 [SMILES: *C1OC(C(=O)O)C(O)C(O)C1O ; *C1OC(CO)C(O)C(O)C1O ; *C1OC(COC2OC(C)C(O)C(O)C2O)C(O)C(O)C1O]

R₂₆ is a carbon atom and together with at least one of R₂₇ and R₂₈ forms a 5- to 8-membered ring, and the one of R₂₇ and R₂₈ that does not form the ring with R₂₆ is selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₂₋₈ alkynyl, CH═CH(C₁₋₈ alkyl), C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂.

R₂₉ is selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₂₋₈ alkynyl, CH═CH(C₁₋₈ alkyl), C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂.

Specifically, the compound of formula III described herein is a compound of formula III-1 or III-2:

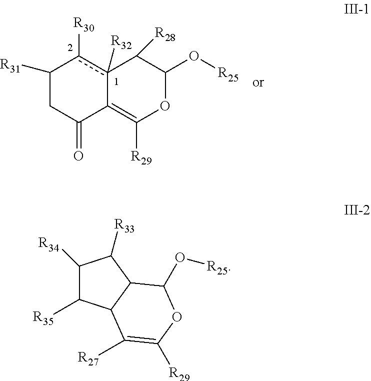

Wherein, R₃₀, R₃₁, R₃₃, R₃₄ and R₃₅ are independently selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₂₋₈ alkynyl, CH═CH(C₁₋₈ alkyl), C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂.

In formula III-1, the bond between carbon atom at positions 1 and 2 is selected from a double bond and a single bond. When in formula III-1 the bond between the carbon atoms at positions 1 and 2 is a double bond, R₃₂ is absent; when in formula III-1 the bond between the carbon atoms at positions 1 and 2 is a single bond, R₃₂ is selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₂₋₈ alkynyl, CH═CH(C₁₋₈ alkyl), C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂.

Specifically, R₂₅ is

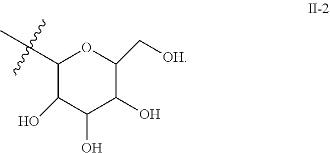 [SMILES: *C1OC(CO)C(O)C(O)C1O]

Specifically, the one of R₂₇ and R₂₈ that does not form the ring with R₂₆ is selected from H, C₁₋₃ alkyl, O(C₁₋₃ alkyl), O(C₁₋₃ cycloalkyl), C₂₋₃ alkenyl, C₂₋₃ alkynyl, CH═CH(C₁₋₃ alkyl), C(C₁₋₃ alkyl)═CH(C₁₋₃ alkyl), C(C₁₋₃ alkyl)═C(C₁₋₃ alkyl)₂, OC(═O)(C₁₋₃ alkyl), C(═O)(C₁₋₃ alkyl), (C₁₋₃ alkyl)CO₂H and CO₂H. More specifically, the one of R₂₇ and R₂₈ that does not form the ring with R₂₆ is selected from H, CH═CH₂ and CO₂H.

Specifically, R₃₀, R₃₁, R₃₃, R₃₄ and R₃₅ are independently selected from H, C₁₋₃ alkyl, O(C₁₋₃ alkyl), C₃₋₆ cycloalkyl, OH, NH₂, CH═CH(C₁₋₃ alkyl), C(C₁₋₃ alkyl)=CH₂, C(C₁₋₃ alkyl)═CH(C₁₋₃ alkyl) and C(C₁₋₃ alkyl)═C(C₁₋₃ alkyl)₂. More specifically, R₃₀, R₃₁, R₃₃, R₃₄ and R₃₅ are independently selected from H, methyl and OH.

Specifically, when the bond between carbon atom at positions 1 and 2 in formula III-1 is a double bond, R₃₂ is absent.

In a specific embodiment of the present invention, the compound of formula III is swertiamarin, sweroside, gentiopicroside, loganic acid or a salt, an isomer or a solvate thereof.

Specifically, the compound or the salt, the isomer or the solvate thereof described herein can be used as the sole active ingredient for controlling or killing mites, and can also be used in combination with other ingredients with the same or different activities for controlling or killing mites.

The compounds described herein can be obtained by extraction from natural plants, such as Herba Taraxaci, Gentiana macrophylla, gentian, Chrysanthemum and the like, and the extraction method can be a conventional method in the art.

The compounds described herein can also be prepared by chemical synthesis or biosynthesis.

Specifically, the product for killing mites described above can be used for therapeutic and/or prophylactic purposes, and also for non-therapeutic and/or non-prophylactic purposes.

Specifically, the mites described above can be one or more of Demodex spp., Dermatophagoides pteronyssinus, Sarcoptes scabiei. and the like. In an embodiment of the present invention, the mites described above are Demodex spp., such as D. folliculorum and D. brevis.

In an embodiment of the present invention, the product for killing mites described above is a pharmaceutical composition.

Specifically, the pharmaceutical composition described above further comprises a pharmaceutically acceptable excipient(s).

Specifically, the pharmaceutical composition described above is used for preventing and/or treating a disease caused by mite infestation.

Specifically, the disease described above may be an ocular disease, a skin disease, an allergic disease and the like.

Specifically, the ocular disease described above may be one or more of blepharitis marginalis, meibomian gland dysfunction, tarsitis, madarosis, abnormal eyelash alignment, glaucoma, cataract, ocular folliculitis, conjunctivitis, blepharoconjunctivitis, pterygium, keratitis, eyelid laxity and ectropion, basal cell carcinoma of eyelid, xerophthalmia, chalazion and the like; the ocular disease may have one or more symptoms selected from: red and itchy eyes, dry eyes, burning eyes, foreign body sensation, photophobia, increased eye discharge, loss of eyelashes, blurred vision, decreased vision and the like.

Specifically, the skin disease described above may be one or more of seborrhoeic dermatitis, acne, acne rosacea, pityriasis folliculorum, perioral dermatitis, demodicosis, gaile, basal cell carcinoma and the like.

Specifically, the allergic disease described above may be one or more of allergic asthma, allergic rhinitis, allergic dermatitis and the like.

Specifically, the pharmaceutical composition described above may be in any dosage form suitable for administration, such as a topical preparation, in particular, an ophthalmic preparation, a skin topical preparation and the like.

Specifically, the ophthalmic preparation described above may be an eye drop, an eye ointment, an ophthalmic gel, an ophthalmic emulsion, an ophthalmic suspension, an ophthalmic film, a collyrium, an intraocular injection and the like.

Specifically, the ophthalmic preparation may comprise a pharmaceutically acceptable excipient(s) such as a pH regulator, a co-solvent, an osmotic pressure regulator, a viscosity regulator, an antioxidant, a bacteriostatic preservative, a buffer, a suspending agent, a local anesthetic, a surfactant, a solubilizer, a wetting agent, an emulsifier, a stabilizer, a filler, a protective agent, a solvent and the like.

Specifically, the skin topical preparation described above may be an aerosol, a powder, a lotion, a tincture, a liniment, a film, an ointment, a gel, a paste, an emulsion and the like.

Specifically, the dosage forms of the pharmaceutical composition described above can be prepared according to conventional production methods in the field of pharmaceuticals.

Specifically, the pharmaceutical composition described above may comprise 0.01%-99.5% (e.g., 0.01%, 0.1%, 0.5%, 1%, 2%, 3%, 4%, 5%, 6%, 7%, 8%, 9%, 10%, 20%, 30%, 40%, 50%, 60%, 70%, 80%, 90%, 95%, 99% or 99.5%) by weight of the active ingredient.

Specifically, the pharmaceutical composition described above can be used in humans, and can also be used as a veterinary drug in non-human animals, such as non-human mammals, e.g., companion animals (e.g., dogs, cats, rabbits, mice, etc.), livestock animals (e.g., horses, cows, sheep, pigs, dogs, rabbits, etc.) and the like.

In another embodiment of the present invention, the product for killing mites described above is a cosmetic product.

Specifically, the cosmetic product described above further comprises a cosmetically acceptable excipient(s).

Specifically, the cosmetic product described above may be a cosmetic product used for the face, such as a facial cleanser, a soap, a skin softener, a toner, a skin care lotion, a jelly lotion, a facial cream, a sunscreen cream, an essence, a facial mask, a gel, a foundation and a scrub.

Specifically, the cosmetic product described above may be a cosmetic product used for parts of the body other than the face, such as a neck cream, a shampoo, a shower gel, a hair conditioner, a body lotion, a scrub and the like.

Specifically, the cosmetic product described above may also be a cosmetic product used for the eyes and periocular region, such as an eye cream, a mascara, an eyeliner powder, a cream eyeliner, an eyeliner pencil, an eye shadow powder, an eye shadow cream, an eyebrow pencil, a brow powder and the like.

Specifically, the various forms of the cosmetic product described above can be prepared according to conventional production methods in the field of cosmetics.

Specifically, the cosmetic product described above may comprise 0.01%-99.5% (e.g., 0.01%, 0.1%, 0.5%, 1%, 2%, 3%, 4%, 5%, 6%, 7%, 8%, 9%, 10%, 20%, 30%, 40%, 50%, 60%, 70%, 80%, 90%, 95%, 99% or 99.5%) by weight of the active ingredient.

In another embodiment of the present invention, the product for killing mites described above is an acaricide which can be used for killing and controlling mites that may live in articles in the living environment (e.g., pillow case, pillow inner, bed sheet, bedding, mattress, clothing, carpet, cushion, sofa, summer sleeping mat, plush toy, air conditioner and the like).

Specifically, the acaricide described above may comprise any suitable excipient that can achieve the desired properties.

Specifically, the acaricide described above may be in a form of a spray, a lotion, a paster, a small packet and the like.

Specifically, the various forms of the acaricide described above can be prepared according to conventional production methods in the field of daily care products.

Specifically, the acaricide described above may comprise 0.01%-99.5% (e.g., 0.01%, 0.1%, 0.5%, 1%, 2%, 3%, 4%, 5%, 6%, 7%, 8%, 9%, 10%, 20%, 30%, 40%, 50%, 60%, 70%, 80%, 90%, 95%, 99% or 99.5%) by weight of the active ingredient.

The present invention further provides a method for preventing and/or treating a disease caused by mite infestation, comprising administering to a subject in need thereof the compound or the salt, the isomer or the solvate thereof described above (in particular, taraxasterol, taraxerol, taraxerone, roburic acid, taraxasterol acetate, taraxeryl acetate, lupenone, or the salt, the isomer or the solvate thereof; diosmetin or the salt, the isomer or the solvate thereof; or swertiamarin, sweroside, gentiopicroside, loganic acid or the salt, the isomer or the solvate thereof) or the pharmaceutical composition described above.

Specifically, the disease described above may be an ocular disease, a skin disease, an allergic disease and the like.

Specifically, the ocular disease described above may be one or more of blepharitis marginalis, meibomian gland dysfunction, tarsitis, madarosis, abnormal eyelash alignment, glaucoma, cataract, ocular folliculitis, conjunctivitis, blepharoconjunctivitis, pterygium, keratitis, eyelid laxity and ectropion, basal cell carcinoma of eyelid, xerophthalmia, chalazion and the like; the ocular disease may have one or more symptoms selected from: red and itchy eyes, dry eyes, burning eyes, foreign body sensation, photophobia, increased eye discharge, loss of eyelashes, blurred vision, decreased vision and the like.

Specifically, the skin disease described above may be one or more of seborrhoeic dermatitis, acne, acne rosacea, pityriasis folliculorum, perioral dermatitis, demodicosis, gaile, basal cell carcinoma and the like.

Specifically, the allergic disease described above may be one or more of allergic asthma, allergic rhinitis, allergic dermatitis and the like.

Specifically, the subject described above can be any animal receiving the prophylaxis and/or treatment, in particular, a mammal, such as a human, a cat, a dog, a rabbit, a mouse, a horse, a cow, a sheep, a pig and the like. In an embodiment of the present invention, the subject described above is a human; and in another embodiment of the present invention, the subject described above is a non-human animal.

In an embodiment of the present invention, the method described above is a method for treating and/or preventing an ocular disease caused by mites, comprising administering to eyes of a subject in need thereof the compound or the salt, the isomer or the solvate thereof described above (in particular, taraxasterol, taraxerol, taraxerone, roburic acid, taraxasterol acetate, taraxeryl acetate, lupenone, or the salt, the isomer or the solvate thereof; diosmetin or the salt, the isomer or the solvate thereof; or swertiamarin, sweroside, gentiopicroside, loganic acid or the salt, the isomer or the solvate thereof) or the pharmaceutical composition described above.

Specifically, the ophthalmic administration described above may be an administration in a form of an eye drop, an eye ointment, an ophthalmic gel, an ophthalmic emulsion, an ophthalmic suspension, an ophthalmic film, a collyrium or an intraocular injection.

Specifically, the dose of the compounds or the pharmaceutical composition described above may vary according to the route of administration, the age and body weight of the subject, the disease to be treated in the subject and the severity, etc., and may be administered in one or more doses.

It is found by comparison that the compounds described herein can reduce the survival time of mites as compared with the control group. In particular, taraxerol, diosmetin, gentiopicroside and taraxasterol acetate can significantly shorten the survival time of mites, and can be used for preparing a product (such as a drug, a cosmetic product, a daily product and the like) for killing or controlling mites.

### DETAILED DESCRIPTION

Unless otherwise defined, all scientific and technical terms used herein have the same meaning as commonly understood by those skilled in the art to which the present invention relates.

In the present invention, the term “animal” generally refers to vertebrates, in particular, mammals, including humans. The term “non-human animal” refers to any vertebrate except human beings, in particular, mammals. In some embodiments of the present invention, the non-human animal described herein is a domestic animal, i.e., an animal raised and domesticated by humans with artificially controlled reproduction for purposes such as food, labor, fur, companionship and experiment, such as an economic animal, a companion animal and a laboratory animal. The economic animal may be, for example, a livestock animal such as a pig, a cow, a sheep, a horse, a donkey, a fox, a raccoon dog, a mink, a camel and the like. The companion animal may be, for example, a dog, a cat, a rabbit, a murine (such as a guinea pig, a hamster, a gerbil, a chinchilla, a squirrel, etc.) and the like. The laboratory animal may be, for example, a monkey, a dog, a rabbit, a cat, a murine (such as a rat and a mouse) and the like.

The technical schemes of the present invention will be clearly and completely described below with reference to the examples of the present invention, and it is obvious that the described examples are only a part of the examples of the present invention but not all of them. Based on the examples of the present invention, all other examples obtained by those of ordinary skill in the art without creative work shall fall within the protection scope of the present invention.

### Example 1: Preliminary Screening In Vitro of Compounds for Effect on Survival Time of Demodex Mites

### Methodology:

### 1. Monomer Compound

The following monomer compounds were selected. The names and molecular formulas are shown in Table 1 below.

The monomer compounds were purchased from Chengdu Herbpurify Co., Ltd., as standard references.

TABLE 1
Names and molecular formulas of monomer compounds

| | | | Molecular |
|---|---|---|---|
| No. | Chinese name | English name | formula |
| 1 | [symbol] | Taraxasterol | C30H50O |
| 2 | [symbol] | Taraxerol | C30H50O |
| 3 | [symbol] | Gentiopicroside | C16H20O9 |
| 4 | [symbol] | Taraxerone | C30H48O |
| 5 | [symbol] | Diosmetin | C16H12O6 |
| 6 | [symbol] | Sweroside | C16H22O9 |
| 7 | [symbol] | Roburic acid | C30H48O2 |
| 8 | [symbol] | Taraxasterol acetate | C32H52O2 |
| 9 | [symbol] | Taraxeryl acetate | C32H52O2 |
| 10 | [symbol] | Lupenone | C30H48O |
| 11 | [symbol] | Swertiamarine | C16H22O10 |
| 12 | [symbol] | Loganic acid | C16H24O10 |

### 2. Preparation of Solutions

The powders of the reference compounds shown in Table 1 were separately dissolved in dimethyl sulfoxide (DMSO), and then diluted with sterilized double-distilled water to make the final concentration of DMSO 10%, which could not shorten the survival time of Demodex mites as per a controlled study. The negative control group received DMSO mixed with sterilized water at a final concentration of 10%.

### 3. In-Vitro Culture of Demodex Mites

35 μL of each solution was added to a glass slide, and the survival of the mites was observed under an optical microscope every 2 hours. During the experiment, the Demodex mites were observed by two skilled operators separately for the activities (bodies, limbs and the like) through the microscope to determine whether they were dead or not. If the two operators gave different results, the survival status was determined independently by a third skilled operator. The in-vitro culture was conducted in a climatic chamber at a temperature of 20° C. and a humidity of 96%. During the observation, the glass slides were transported in a wet box to ensure the high humidity.

### 4. Statistics

In this experiment, statistical analysis was conducted using SPSS22.0 statistical software, normality test was conducted using Shapiro-Wilk test, and homogeneity of variance test was conducted using Bartlett's test. The data with normal distribution and homogeneous variance were subjected to One-way ANOVA, and when there was statistical significance, Bonferroni's method was used for pairwise comparison among the groups; when the data did not converge to normal distribution, the Kruskal-Wallis test was adopted for statistical analysis with a test criterion a of 0.05.

### 5. Results:

Effects of compounds on survival time of D. folliculorum The in-vitro culture of the Demodex mites was conducted in an environment at a humidity of 96% and a temperature of 20° C. The survival of the Demodex mites was observed dynamically and the survival time was recorded. The results are shown in Table 2. Compared with the negative control group receiving 10% DMSO, the survival time of the Demodex mites in vitro was significantly shorter in the taraxerol group (37.91±20.96 vs. 80.79±25.34, P=0.006), the diosmetin group (35.92±14.82 vs. 80.79±25.34, P=0.001), the taraxasterol acetate group (37.91±20.96 vs. 80.79±25.34, P<0.001) and the gentiopicroside group (37.31±36.06 vs. 80.79±25.34, P<0.001), while the survival time of the Demodex mites in the groups of other monomer compounds did not show significant differences (P>0.05) as compared with the control group.

TABLE 2
Effects of compounds on survival time of D. folliculorum in vitro

| | | | Number of |
|---|---|---|---|
| | | Survival time | Demodex |
| No. | Monomer compound | (hours) | mites (N) |
| Control | 10% DMSO | 80.79 ± 25.34 | 36 |
| 1 | Taraxasterol | 68.28 ± 31.30 | 6 |
| 2 | Taraxerol | 37.91 ± 20.96** | 11 |
| 3 | Gentiopicroside | 37.31 ± 36.06*** | 15 |
| 4 | Taraxerone | 62.06 ± 28.97 | 8 |
| 5 | Diosmetin | 35.92 ± 14.82** | 10 |
| 6 | Loganic acid | 61.56 ± 22.84 | 8 |
| 7 | Sweroside | 60.79 ± 25.98 | 7 |
| 8 | Roburic acid | 63.41 ± 12.20 | 9 |
| 9 | Taraxasterol acetate | 34.14 ± 22.96*** | 13 |
| 10 | Taraxeryl acetate | 70.57 ± 27.45 | 12 |
| 11 | Lupenone | 66.25 ± 31.22 | 5 |
| 12 | Swertiamarin | 59.68 ± 10.97 | 7 |

Multiple comparisons were conducted using Duncan's test; as compared with the control group,
P*: p < 0.05,
P**: p < 0.01,
P***: p < 0.001.

The above description is only for the purpose of illustrating the preferred example of the present invention, and is not intended to limit the scope of the present invention. Any modifications, equivalents and the like made without departing from the spirit and principle of the present invention shall fall within the protection scope of the present invention.

The foregoing examples and methods described herein may vary based on the abilities, experience, and preferences of those skilled in the art.

The certain order in which the procedures are described in the present invention does not constitute any limitation to the order of the procedures.

1. Use of a compound of formula I or a salt, an isomer or a solvate thereof in preparing a product for controlling or killing mites, wherein the compound of formula I is: 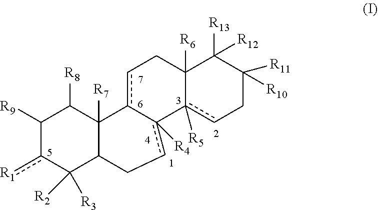
wherein, R₁ is selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₂₋₈ alkynyl, CH═CH₂, CH═CH(C₁₋₈ alkyl), C(C₁₋₈ alkyl)=CH₂, C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂, or when the bond between R₁ and carbon atom at position 5 is a double bond, R₁ is selected from O and S;
R₂, R₃, R₆, R₇, R₈ and R₉ are independently selected from H, C₁₋₈ alkyl, O(C₁₋₈ alkyl), NH(C₁₋₈ alkyl), N(C₁₋₈ alkyl)₂, C₃₋₁₁ cycloalkyl, O(C₁₋₈ cycloalkyl), OH and NH₂;
R₄ is selected from H, C₁₋₈ alkyl, O(C₁₋₈ alkyl), NH(C₁₋₈ alkyl), N(C₁₋₈ alkyl)₂, C₃₋₁₁ cycloalkyl, O(C₁₋₈ cycloalkyl), OH and NH₂, or when the bond between carbon atoms at positions 1 and 4 is a double bond, R₄ is absent;
R₅ is selected from H, C₁₋₈ alkyl, O(C₁₋₈ alkyl), NH(C₁₋₈ alkyl), N(C₁₋₈ alkyl)₂, C₃₋₁₁ cycloalkyl, O(C₁₋₈ cycloalkyl), OH and NH₂, or when the bond between carbon atoms at positions 2 and 3 is a double bond, R₅ is absent;
the bond between carbon atoms at positions 6 and 7 is a double bond or single bond;
R₁₀ and R₁₃ are independently selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₁₋₈ alkynyl, CH═CH(C₁₋₈ alkyl), CH═CH₂, C(C₁₋₈ alkyl)=CH₂, C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂; and
R₁₁ and R₁₂ are independently selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₁₋₈ alkynyl, CH═CH(C₁₋₈ alkyl), C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂, or R₁₁ and R₁₂ are carbon atoms and together with carbon atoms therebetween form a 5- to 8-membered ring, wherein H on any carbon atom on the ring may be substituted with C₁₋₈ alkyl, O(C₁₋₈ alkyl), NH(C₁₋₈ alkyl), N(C₁₋₈ alkyl)₂, C₃₋₁₁ cycloalkyl, O(C₁₋₈ cycloalkyl), OH, NH₂, C₂₋₈ alkenyl, C₂₋₈ alkynyl, CH═CH₂, C(C₁₋₈ alkyl)=CH₂, CH═CH(C₁₋₈ alkyl), C(C₁₋₈ alkyl)═CH(C₁₋₈ alkyl) or C(C₁₋₈ alkyl)═C(C₁₋₈ alkyl)₂.

2. The use according to claim 1, wherein the compound of formula I is a compound of formula I-1: 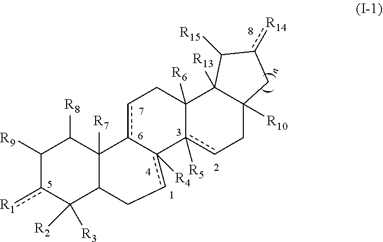
wherein, when the bond between R₁₄ and carbon atom at position 8 is a single bond, R₁₄ is selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₂₋₈ alkynyl, CH═CH(C₁₋₈ alkyl), C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂, and hydrogen atom on the carbon atom at position 8 is optionally substituted with R₁₄; when the bond between R₁₄ and the carbon atom on position 8 is a double bond, R₁₄ is selected from O, S, CH₂, CH(C₁₋₈ alkyl) and C(C₁₋₈ alkyl)₂;
R₁₅ is selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₂₋₈ alkynyl, CH═CH(C₁₋₈ alkyl), C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂; and
n is an integer of 1-4, such as 1, 2, 3 or 4.

3. The use according to claim 1, wherein R₁ is selected from H, C₁₋₃ alkyl, OH, OC(═O)(C₁₋₃ alkyl), (C₁₋₃ alkyl)CO₂H, CF₃, CHF₂ and CH₂F, or when the bond between R₁ and carbon atom at position 5 is a double bond, R₁ is O;
R₂, R₃, R₆, R₇, R₅ and R₉ are independently selected from H, C₁₋₃ alkyl, O(C₁₋₃ alkyl), C₃₋₆ cycloalkyl, OH and NH₂;
the bond between carbon atoms at positions 1 and 4 is a single bond, and R₄ is selected from H, C₁₋₃ alkyl, O(C₁₋₃ alkyl), C₃₋₆ cycloalkyl, O(C₁₋₃ cycloalkyl) and OH;
the bond between carbon atoms at positions 2 and 3 is a single bond, and R₅ is selected from H, C₁₋₃ alkyl, O(C₁₋₃ alkyl), C₃₋₆ cycloalkyl, O(C₁₋₃ cycloalkyl) and OH;
the bond between carbon atoms at positions 6 and 7 is a single bond;
R₁₀ and R₁₃ are independently selected from H, C₁₋₃ alkyl, O(C₁₋₃ alkyl), C₃₋₆ cycloalkyl, OH and NH₂;
when bond between R₁₄ and the carbon atom at position 8 is a single bond, R₁₄ is selected from H, C₁₋₃ alkyl, O(C₁₋₃ alkyl), C₃₋₆ cycloalkyl, OH and NH₂, and similarly, the hydrogen atom on the carbon atom at position 8 is substituted with R₁₄; when bond between R₁₄ and the carbon atom at position 8 is a double bond, R₁₄ is selected from CH₂, CH(C₁₋₃ alkyl) and C(C₁₋₃ alkyl)₂; and
R₁₅ is selected from H, C₁₋₃ alkyl, O(C₁₋₃ alkyl), C₃₋₆ cycloalkyl, OH, NH₂, CH═CH(C₁₋₃ alkyl), C(C₁₋₃ alkyl)=CH₂, C(C₁₋₃ alkyl)═CH(C₁₋₃ alkyl) and C(C₁₋₃ alkyl)═C(C₁₋₃ alkyl)₂; preferably, R₁₅ is selected from H, C₁₋₃ alkyl and C(CH₃)═CH₂.

4. The use according to claim 1, wherein the compound of formula I is taraxasterol, taraxerol, taraxerone, roburic acid, taraxasterol acetate, taraxeryl acetate, lupenone, or a salt, an isomer or a solvate thereof.

5. Use of a compound of formula II or a salt, an isomer or a solvate thereof in preparing a product for controlling or killing mites, wherein the compound of formula II is: 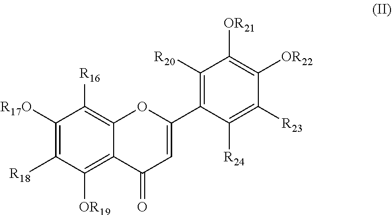
wherein, R₁₆, R₁₈, R₂₀, R₂₃ and R₂₄ are independently selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₂₋₈ alkynyl, CH═CH(C₁₋₈ alkyl), C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂; and
R₁₇, R₁₉, R₂₁ and R₂₂ are independently selected from H, C₁₋₈ alkyl, O(C₁₋₈ alkyl), C₃₋₁₁ cycloalkyl, aryl, heteroaryl, C₃₋₁₁ heterocycloalkyl, O(C₁₋₈ cycloalkyl) and a group of formula II-1, II-2 or II-3:

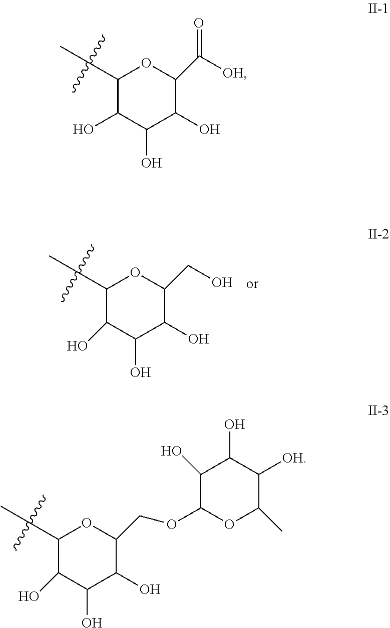 [SMILES: *C1OC(C(=O)O)C(O)C(O)C1O ; *C1OC(CO)C(O)C(O)C1O ; *C1OC(COC2OC(C)C(O)C(O)C2O)C(O)C(O)C1O]

6. The use according to claim 5, wherein R₁₆, R₁₈, R₂₀, R₂₃ and R₂₄ are independently selected from H, halogen, C₁₋₃ alkyl, O(C₁₋₃ alkyl), NH(C₁₋₃ alkyl), N(C₁₋₃ alkyl)₂, C₃₋₁₁ cycloalkyl, OH, NH₂, OC(═O)(C₁₋₃ alkyl), C(═O)(C₁₋₃ alkyl), (C₁₋₃ alkyl)CO₂H, CO₂H, CN, CF₃, CHF₂, CH₂F, NO₂, C₂₋₃ alkenyl, C₂₋₃ alkynyl, CH═CH(C₁₋₃ alkyl), C(C₁₋₃ alkyl)═CH(C₁₋₃ alkyl) and C(C₁₋₃ alkyl)═C(C₁₋₃ alkyl)₂; and
R₁₇, R₁₉, R₂₁ and R₂₂ are independently selected from H, C₁₋₃ alkyl, O(C₁₋₃ alkyl) and a group of formula II-1 or II-2.

7. The use according to claim 5, wherein the compound of formula II is diosmetin or a salt, an isomer or a solvate thereof.

8. Use of a compound of formula III or a salt, an isomer or a solvate thereof in preparing a product for controlling or killing mites, wherein the compound of formula III is: 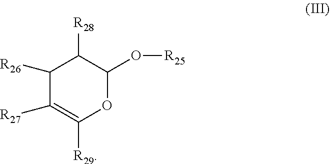
wherein R₂₅ is selected from 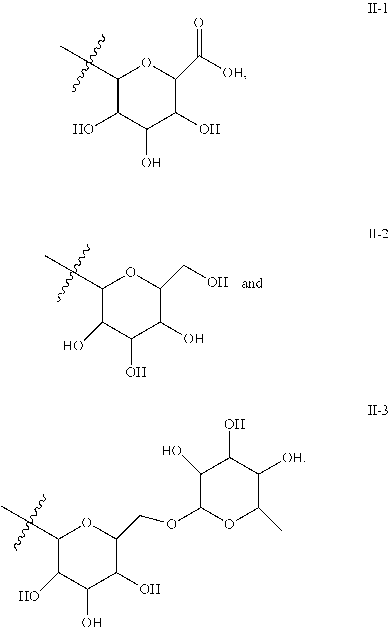 [SMILES: *C1OC(C(=O)O)C(O)C(O)C1O ; *C1OC(CO)C(O)C(O)C1O ; *C1OC(COC2OC(C)C(O)C(O)C2O)C(O)C(O)C1O]
R₂₆ is a carbon atom and together with at least one of R₂₇ and R₂₈ forms a 5- to 8-membered ring, and the one of R₂₇ and R₂₈ that does not form the ring with R₂₆ is selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₂₋₈ alkynyl, CH═CH(C₁₋₈ alkyl), C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂; and
R₂₉ is selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₂₋₈ alkynyl, CH═CH(C₁₋₈ alkyl), C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂.

9. The use according to claim 8, wherein the compound of formula III is a compound of formula III-1 or III-2: 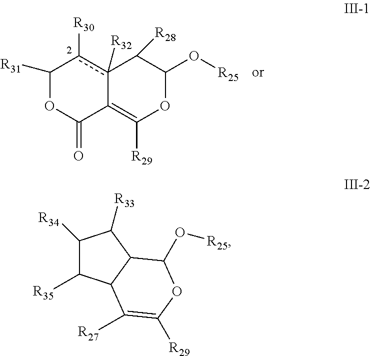
wherein, R₃₀, R₃₁, R₃₃, R₃₄ and R₃₅ are independently selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₂₋₈ alkynyl, CH═CH(C₁₋₈ alkyl), C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂;
in formula III-1, the bond between carbon atom at positions 1 and 2 is selected from a double bond and a single bond,
when in formula III-1 the bond between carbon atoms at positions 1 and 2 is a double bond, R₃₂ is absent; when in formula III-1 the bond between carbon atoms at positions 1 and 2 is a single bond, R₃₂ is selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₂₋₈ alkynyl, CH═CH(C₁₋₈ alkyl), C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂.

10. The use according to claim 9, wherein R₂₅ is selected from 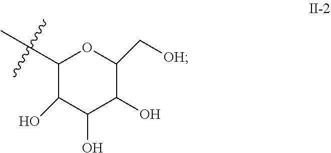 [SMILES: *C1OC(CO)C(O)C(O)C1O]
the one of R₂₇ and R₂₈ that does not form the ring with R₂₆ is selected from H, C₁₋₃ alkyl, O(C₁₋₃ alkyl), O(C₁₋₃ cycloalkyl), C₂₋₃ alkenyl, C₂₋₃ alkynyl, CH═CH(C₁₋₃ alkyl), C(C₁₋₃ alkyl)═CH(C₁₋₃ alkyl), C(C₁₋₃ alkyl)═C(C₁₋₃ alkyl)₂, OC(═O)(C₁₋₃ alkyl), C(═O)(C₁₋₃ alkyl), (C₁₋₃ alkyl)CO₂H and CO₂H;
R₃₀, R₃₁, R₃₃, R₃₄ and R₃₅ are independently selected from H, C₁₋₃ alkyl, O(C₁₋₃ alkyl), C₃₋₆ cycloalkyl, OH, NH₂, CH═CH(C₁₋₃ alkyl), C(C₁₋₃ alkyl)=CH₂, C(C₁₋₃ alkyl)═CH(C₁₋₃ alkyl) and C(C₁₋₃ alkyl)═C(C₁₋₃ alkyl)₂; preferably, R₃₀, R₃₁, R₃₃, R₃₄ and R₃₅ are independently selected from H, methyl and OH; and
in formula III-1, the bond between carbon atom at positions 1 and 2 is a double bond, and R₃₂ is absent.

11. The use according to claim 8, wherein the compound of formula III is swertiamarin, sweroside, gentiopicroside, loganic acid or a salt, an isomer or a solvate thereof.

12. The use according to claim 1, wherein the mites are one or more of Demodex spp., Dermatophagoides pteronyssinus and Sarcoptes scabiei.

13. The use according to claim 1, wherein the product for controlling or killing mites is a pharmaceutical composition, a cosmetic product or an acaricide.

14. The use according to claim 13, wherein the pharmaceutical composition is a pharmaceutical composition for preventing and/or treating a disease caused by mite infestation;
the disease is selected from: an ocular disease, a skin disease and an allergic disease;
preferably, the ocular disease is selected from: blepharitis marginalis, meibomian gland dysfunction, tarsitis, madarosis, abnormal eyelash alignment, glaucoma, cataract, ocular folliculitis, conjunctivitis, blepharoconjunctivitis, pterygium, keratitis, eyelid laxity and ectropion, basal cell carcinoma of eyelid, xerophthalmia and chalazion;
preferably, the skin disease is selected from: seborrhoeic dermatitis, acne, acne rosacea, pityriasis folliculorum, perioral dermatitis, demodicosis, gaile and basal cell carcinoma; and
preferably, the allergic disease is selected from: allergic asthma, allergic rhinitis and allergic dermatitis.

15. The use according to claim 13, wherein the pharmaceutical composition is a topical preparation, preferably an ophthalmic preparation or a skin topical preparation;
preferably, the ophthalmic preparation is selected from: an eye drop, an eye ointment, an ophthalmic gel, an ophthalmic emulsion, an ophthalmic suspension, an ophthalmic film, a collyrium and an intraocular injection;
preferably, the skin topical preparation is selected from: an aerosol, a powder, a lotion, a tincture, a liniment, a film coating agent, an ointment, a gel, a paste and an emulsion.

16. The use according to claim 13, wherein the pharmaceutical composition is a human pharmaceutical composition or a veterinary pharmaceutical composition.

17. The use according to claim 13, wherein the cosmetic product is in a form selected from: a facial cleanser, a soap, a skin softener, a toner, a skin care lotion, a jelly lotion, a facial cream, a sunscreen cream, an essence, a facial mask, a gel, a foundation, a scrub, a neck cream, a shampoo, a shower gel, a hair conditioner, a body lotion, an eye cream, a mascara, an eyeliner powder, a cream eyeliner, an eyeliner pencil, an eye shadow powder, an eye shadow cream, an eyebrow pencil and a brow powder.

18. The use according to claim 13, wherein the acaricide is in a form selected from: a spray, a lotion, a paster and a small packet.

19. A method for treating and/or preventing an ocular disease caused by mites, comprising administering to eyes of a subject in need thereof a compound of formula I, wherein the compound of formula I is: 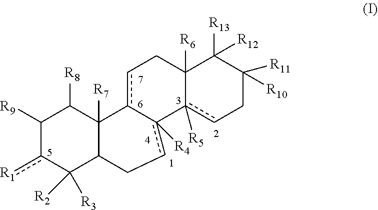
wherein, R₁ is selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₂₋₈ alkynyl, CH═CH₂, CH═CH(C₁₋₈ alkyl), C(C₁₋₈ alkyl)=CH₂, C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂, or when the bond between R₁ and carbon atom at position 5 is a double bond, R₁ is selected from O and S;
R₂, R₃, R₆, R₇, R₈ and R₉ are independently selected from H, C₁₋₈ alkyl, O(C₁₋₈ alkyl), NH(C₁₋₈ alkyl), N(C₁₋₈ alkyl)₂, C₃₋₁₁ cycloalkyl, O(C₁₋₈ cycloalkyl), OH and NH₂;
R₄ is selected from H, C₁₋₈ alkyl, O(C₁₋₈ alkyl), NH(C₁₋₈ alkyl), N(C₁₋₈ alkyl)₂, C₃₋₁₁ cycloalkyl, O(C₁₋₈ cycloalkyl), OH and NH₂, or when the bond between carbon atoms at positions 1 and 4 is a double bond, R₄ is absent;
R₅ is selected from H, C₁₋₈ alkyl, O(C₁₋₈ alkyl), NH(C₁₋₈ alkyl), N(C₁₋₈ alkyl)₂, C₃₋₁₁ cycloalkyl, O(C₁₋₈ cycloalkyl), OH and NH₂, or when the bond between carbon atoms at positions 2 and 3 is a double bond, R₅ is absent;
the bond between carbon atoms at positions 6 and 7 is a double bond or single bond;
R₁₀ and R₁₃ are independently selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₁₋₈ alkynyl, CH═CH(C₁₋₈ alkyl), CH═CH₂, C(C₁₋₈ alkyl)=CH₂, C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂; and
R₁₁ and R₁₂ are independently selected from H, halogen, C₁₋₈ alkyl, O(C₁₋₈ alkyl), S(C₁₋₈ alkyl), NH(C₁₋₈ alkyl), N(C₁₋₈ alkyl)₂, C₃₋₁₁ cycloalkyl, aryl, heteroaryl, C₃₋₁₁ heterocycloalkyl, O(C₁₋₈ cycloalkyl), S(C₁₋₈ cycloalkyl), NH(C₁₋₈ cycloalkyl), N(C₁₋₈ cycloalkyl)(C₁₋₈ alkyl), OH, NH₂, SH, SO₂(C₁₋₈ alkyl), C₂₋₈ alkenyl, C₁₋₈ alkynyl, CH═CH(C₁₋₈ alkyl), C(C₁₋₈ alkyl)═CH(C₁₋₈ alkyl), C(C₁₋₈ alkyl)═C(C₁₋₈ alkyl)₂, OC(═O)(C₁₋₈ alkyl), C(═O)(C₁₋₈ alkyl), (C₁₋₈ alkyl)CO₂H, CO₂H, CN, CF₃, CHF₂, CH₂F, NO₂, C(═O)NH(C₁₋₈ alkyl), C(═O)N(C₁₋₈ alkyl)₂, N(C₁₋₈ alkyl)C(═O)NH(C₁₋₈ alkyl), N(C₁₋₈ alkyl)C(═O)N(C₁₋₈ alkyl)₂, NHC(═O)NH(C₁₋₈ alkyl), NHC(═O)N(C₁₋₈ alkyl)₂, NHC(═O)NH₂, N(C₁₋₈ alkyl)SO₂NH(C₁₋₈ alkyl), N(C₁₋₈ alkyl)SO₂N(C₁₋₈ alkyl)₂, NHSO₂NH(C₁₋₈ alkyl) and NHSO₂N(C₁₋₈ alkyl)₂, or R₁₁ and R₁₂ are carbon atoms and together with carbon atoms therebetween form a 5- to 8-membered ring, wherein H on any carbon atom on the ring may be substituted with C₁₋₈ alkyl, O(C₁₋₈ alkyl), NH(C₁₋₈ alkyl), N(C₁₋₈ alkyl)₂, C₃₋₁₁ cycloalkyl, O(C₁₋₈ cycloalkyl), OH, NH₂, C₂₋₈ alkenyl, C₂₋₈ alkynyl, CH═CH₂, C(C₁₋₈ alkyl)=CH₂, CH═CH(C₁₋₈ alkyl), C(C₁₋₈ alkyl)═CH(C₁₋₈ alkyl) or C(C₁₋₈ alkyl)═C(C₁₋₈ alkyl)₂.
